(19) United States
(12) Patent Application Publication
(10) Pub. No.: US 2024/0288439 A1
(43) Pub. Date: Aug. 29, 2024

(54) METHOD AND DEVICE FOR SPECTROMETRIC ANALYSIS

(71) Applicant: Bruker Daltonics GmbH & Co. KG, Bremen (DE)

(72) Inventors: Tanja BIEN, Bremen (DE); Andreas HAASE, Bremen (DE); Jens BOßMEYER, Bremen (DE)

(21) Appl. No.: 18/583,073

(22) Filed: Feb. 21, 2024

(30) Foreign Application Priority Data
Feb. 23, 2023 (DE) 102023104393.5

(51) Int. Cl. G01N 33/68; H01J 49/00; H01J 49/16

(52) U.S. Cl. CPC G01N 33/6851; H01J 49/0004; H01J 49/164

(57) ABSTRACT

Devices and methods for the spectrometric analysis of sample material located in an ablation area on a sample support are disclosed, including a mode of operation which comprises: (i) locating the ablation area on the sample support and determining an ablation area dimension between opposing boundaries of the ablation area; (ii) beam-assisted sampling from the ablation area, e.g., using MALDI, and mass analyzing, e.g., using an IMS-QoTOF analyzer, the ablated and/or desorbed and ionized sample material, wherein a beam impingement region, which is selected no larger than the dimension of the ablation area, is moved within the boundaries of the ablation area while performing ablation and/or desorption operations and an extension of the beam impingement region is changed at least once; and (iii) combining the molecular information obtained by the ablation and/or desorption operations from the ablation area into a single spectral dataset.

### FIELD OF THE INVENTION

The invention relates to devices and methods for the spectrometric analysis of sample material located in an ablation area on a sample support. The ablation areas are identified and molecules of the sample material taken from the ablation areas are transferred to the gas phase and then subjected to an analysis, concluding with a mass analysis.

### BACKGROUND TO THE INVENTION

The Prior Art is explained below with reference to a special aspect. This is not to be understood as a limitation, however. Useful further developments and modifications of what is known from the Prior Art can also be applied above and beyond the comparatively narrow scope of this introduction, and will easily be evident to practitioners skilled in the art in this field after reading the following disclosure.

The Prior Art essentially describes two ways of sampling and mass analyzing sample material from an ablation area of interest on a sample support for the purpose of spectrometric molecular weight determination. Either the sample material is scanned in a targeted manner within the limits of the ablation area, while surrounding areas on the sample support are not taken into account, or the material is scanned in a grid-like manner over an entire area of the sample support and the spatially-resolved data obtained, which can be spatially assigned only to the scanning area, is combined during post-processing. Scanning beams with a uniform and fixed size are used in both embodiments.

This approach does have its shortcomings, however. If the scanning beam is too large, the material outside the limits of the scanning area of interest will likely be sampled and mass analyzed together with the actual sample material of interest (mixed spectrum). The specificity of the spectral data suffers as a result. On the other hand, if the scanning beam is too small, sampling an ablation area beyond the limits can be avoided, but data collection becomes significantly more complex if the molecular content of the entire ablation area is to be taken into account when obtaining the spectral data for reasons of completeness or measurement sensitivity. The effects of this are particularly serious if the shape and contour of the scanning beam at the point of impingement on the sample support differs substantially from the shape and contour of the ablation area, as can be the case when examining individual biological cells, for example if their shape is very irregular.

Some documents of the Prior Art that may have aspects in common with the approaches explained above are briefly discussed below:

Patent publication WO 2005/079360 A2 relates to an arrangement of optical devices for the rapid design of laser profiles used for desorption and/or ionization sources in analytical mass spectrometry. In particular, a user-defined laser pattern on the sample support is presented, which can be changed to different sizes or shapes for subsequent laser pulses on a microsecond time scale.

Patent EP 1 829 081 B1 claims, amongst other things, a mass spectrometer with a laser, an ion source or imaging device which has a target region, sample surface or target plate provided therein, and a device for controlling the size of a laser beam which, during operation, is directed at the target region, sample surface or target plate. The device may comprise: (a) one or more zoom lenses, (b) at least one beam splitter for splitting one or more laser beams into a first laser beam and a second laser beam, and a means to at least partially or completely overlap the first and second laser beams, or (c) a programmable mirror array or a digital micromirror array which comprises a plurality of individually controllable pixel or mirror elements. Means to control the pixel or mirror elements are provided in order to focus laser light onto the target region, sample surface or target plate.

Patent publication US 2007/0141718 A1 discloses techniques for reducing scanning times in mass spectral tissue imaging studies. According to a first technique, a limit for the tissue image is defined that closely approximates the margins of a tissue sample. In a second technique, low resolution scanning identifies one or more areas of interest within the tissue sample, and the identified areas of interest are subsequently scanned at increased resolution.

The patent publication DE 10 2007 060 438 A1 (corresponds to US 2010/0255531 A1) relates to the examination of individual cells from bodily fluids, swabs or tissues. The cells are deposited on a mass spectrometric sample support, without any overlap as far as possible, the position coordinates of the cells are determined, then the sample support is coated with a layer of small crystals of a matrix substance, the sample support is positioned in accordance with the position coordinates by means of the movement device of a mass spectrometer prior to recording the mass spectra of the individual cells with ionization of the cell components by matrix assisted laser desorption, and the mass spectra are used for analysis of the type, state or other distinguishing features of the cells.

Patent publication US 2015/0364307 A1 relates to systems and methods for single-cell culture and analysis by means of microscopy and MALDI mass spectrometry. The systems and methods isolate a plurality of cells in a plurality of wells so that a predetermined number of the wells contain only a single cell. The wells enable visual inspection of the cells and subsequent MALDI ionization of molecules from the cells.

Patent publication WO 2018/189365 A1 relates to a method for mass spectrometry (MS) with single-cell imaging by correlating an optical image of a cell sample with an MS image. In particular, the method should allow optical and molecular phenotypes to be tested simultaneously with single-cell resolution.

Patent publication EP 3 460 479 A1 relates to a method for evaluating the quality of preparations of analytical tissue samples for mass spectrometric imaging using a reference sample, which is processed and measured together with the analytical tissue samples, as well as a parts kit for a mass spectrometric imaging experiment containing such a reference sample.

Patent publication US 2021/0333173 A1 relates to systems and methods for high-speed modulation sample imaging, in particular for performing Imaging Mass Cytometry, including analysis of marker atoms by elemental (e.g., atomic) mass spectrometry. Aspects include a sampling system with a femtosecond laser and/or laser scanning and a corresponding method. Systems and methods for co-registration of other imaging modalities with Imaging Mass Cytometry are also explained.

Patent publication CN 113866252 A presents a device and method for analyzing the mass spectrum of a single cell, comprising: a counterelectrode for detecting physical laser parameters, a unit comprising a laser and a control unit, a unit for detecting plus a unit for recording physical laser parameters, a light beam control unit used for controlling the laser to erode specified cells, and a unit used for selectively moving an electrospray capillary tube to the top of any cell or to a mass spectrum injection port.

The study by Tanja Bien et al. (PNAS 2022 Vol. 119, No. 29, e211436511) focuses on mass spectrometric imaging to explore molecular heterogeneity in cell cultures.

In light of the above, there is a need to improve the spatially unresolved sampling and mass analysis of sample material from a limited ablation area, whose shape and/or outline differ substantially from the shape and/or outline of the beam impingement region used for ablation and/or desorption, on a sample support. Further objectives that can be achieved by the invention will be immediately apparent to the person skilled in the art from reading the disclosure below.

### BRIEF SUMMARY OF THE INVENTION

According to a first aspect, the invention relates to a method for the spectrometric analysis of sample material located in an ablation area on a sample support, comprising: (i) locating the ablation area on the sample support and determining a dimension for the ablation area between opposing boundaries of the ablation area; (ii) beam-assisted sampling from the ablation area and mass analyzing the ablated and/or desorbed and ionized sample material, wherein a beam impingement region, which is no larger than the dimension for the ablation area, is moved within the limits of the ablation area while performing ablation and/or desorption operations and an extension of the beam impingement region is changed at least once; and (iii) combining the molecular information obtained from the ablation area by means of the ablation and/or desorption operations into a single spectral dataset.

This disclosure is based on the fact that molecular information spectral data acquisition of sample material from an ablation area can be improved by using more than one beam impingement region, adapted to the shape, outline and dimensions of the ablation area, for sample material impact. In particular, the largest possible beam impingement region can be selected that fits into a continuous partial section of the ablation area, which differs substantially from the shape and/or outline of the beam impingement region, in order to sample as much molecular information as possible from the sample material taken from the ablation area while avoiding spatial displacement of the beam or the sample support. Furthermore, the beam impingement region can be modified in order to extract the molecular information of the partial sections that cannot be sampled with the first extension of the beam impingement region without extending beyond the boundaries of the ablation area, and to take it into account when creating the spectral data set.

Compared to a procedure in which a single adjustment of the beam impingement region is employed, which is geared to the smallest sections, extremities and/or projections of the ablation area, the procedure described above has the advantage that fewer adjustment steps of the beam guiding elements and the sample support translation stage are required, which reduces electro-mechanical wear, in particular, and thus increases the service life of the apparatus and equipment used. Furthermore, sampling the largest possible beam impingement region means that a large amount of molecular information can be retrieved from the sample material using individual beam pulses, which makes better use of the dynamic range of the detector, including the digitizer, in the connected mass analyzer, and reduces electronic noise influences. Moreover, the use of fast-reacting electromechanical and/or optomechanical and/or electrooptical and/or optoacoustic components for changing the beam impingement region during sampling can also result in a time advantage compared to continuous small-scale, section-by-section scanning of the ablation area.

Preferably, partial sections of the ablation area that are sampled before and after modifying the extension of the beam impingement region should not overlap, or at least should not overlap substantially. In this way, unnecessary multiple sampling of a partial section in the ablation area or even irradiation of partial sections of the ablation area that have already been depleted of sample material by previous sampling can be avoided for the most part. The proportion of overlapping surface of partial sections sampled with different impingement region settings is preferably no greater than an upper limit selected from among the group including: no greater than 50 percent, or no greater than 40 percent, or no greater than 30 percent, or no greater than 20 percent, or no greater than 10 percent, or most preferably no greater than 5 percent.

The sample support may be plate-shaped. The sample support may have the dimensions of a standard microtitration plate, e.g., 127.71 millimeters long, 85.43 millimeters wide and 14.10 millimeters thick. The material of the sample support may be conductive, e.g., made of steel; it may also have a glass, ceramic or plastic substrate with a conductive surface coating that carries the sample material. One example is indium tin oxide-coated specimen slides.

The ablation area may have a region of interest of the sample material on the sample support. The ablation area is especially limited and has dimensions that are smaller than those of the sample support. According to the disclosure, a surface of a possible ablation area is no greater than an upper limit, for example, which may be selected from among the group including: less than 10 square millimeters, or less than 1 square millimeter, or less than 100 square micrometers, or less than 10 square micrometers, or less than 1 square micrometer in particular. Preferred ablation surface areas encompass an upper limit and a lower limit, each of which may be selected from among the group including: between 10 square millimeters and 1 square micrometer, or between 1 square millimeter and 1 square micrometer, or between 100 square micrometers and 1 square micrometer, or between 10 square micrometers and 1 square micrometer.

In various embodiments, a single cell on the sample support may be selected as the ablation area and sample material. The single cell may be of human or animal origin. In particular, the single cell may be grown directly on the sample support, e.g., by in-situ cultivation, or deposited there. The single cell may be a tissue cell. The single cell may be taxonomically classified as a prokaryote, e.g., a cell of a bacterial or archaeal species, or eukaryote, e.g., a human, animal, plant, fungal or algal cell. The ablation area may be the nucleus itself, nucleus and cytoplasm or the area around the nucleus of a single cell. The ablation area may have a cell wall or cell membrane as limits, which can be detected and located using a non-mass-analytical imaging modality.

The ablation area can be localized and its dimensions determined using a non-mass-analytical imaging modality. The imaging modality may be non-sample consuming. The imaging modality may comprise microscopic images of the side of the sample support carrying the ablation area. Bright-field microscopy, dark-field microscopy, infrared microscopy, near-field microscopy, phase-contrast microscopy, polarization microscopy and/or fluorescence microscopy may be used. The microscopic image may be captured uniformly from the entire sample support or piece by piece by capturing small image elements, which can then be combined using image processing algorithms to form an overall image of the sample material or sample support. Preferably, an image of an imaging modality is taken from the ablation area or the sample support prior to beam-assisted sampling and mass analysis. Localization may comprise determining the position coordinates of the ablation area on the sample support.

In various embodiments, the ablation area dimension may correspond to the largest possible extension of the beam impingement region on the sample material in the ablation area. The ablation area dimension can be a diameter of a circular surface or an edge length of a square surface. The ablation area dimension is preferably determined in such a way that the underlying surface of the beam is fully congruent with sample material from the ablation area and does not overlap the boundaries of the ablation area. The beam impingement region may correspond to a beam focus; the beam focus may indicate the location of the smallest spatial extent of the beam along its path (beam waist).

The boundaries of the ablation area may correspond to the outlines of a sample material form on the sample support. In the case of single cells, the boundaries may correspond to the outer cell boundaries, characterized by cell membrane or cell wall, for example. Preferably, boundaries of different ablation areas on the sample support do not intersect, or in other words, different ablation areas preferably do not overlap. If the sample material is a tissue section, the boundaries may be single cells within the tissue, e.g., a nerve cell, and/or also physiologically or anatomically significant areas of connected cells, e.g., muscle cell areas.

The beam used for sampling may contain electromagnetic waves. The beam may have coherent light, e.g., laser light. The beam may be emitted in pulsed or continuous mode. The beam may strike the sample material in the ablation area in incident light (in reflection mode) or transmitted light (in transmission mode). The beam may comprise a beam profile with a single intensity peak or a pattern of intensity peaks. Examples for such designs can be found in patent publications DE 10 2004 044 196 A1 (corresponds to US 2006/0071160 A1), DE 10 2005 006 125 A1 (corresponds to US 2006/0186332 A1) and DE 10 2013 018 496 A1 (corresponds to US 2015/0122986 A1) of the applicant. If a pattern is utilized, the beam impingement region may be defined by a geometric outline that can be visualized as closely surrounding all intensity peaks, e.g., a circular line or similar (oval, ellipse, etc.).

A laser beam may have a so-called “flat top” profile at the point of impingement on the sample material. This profile is characterized by a comparatively low-transition box shape. The resulting clear limits of the beam allow a spatially clearly delimited sampling of sample material, regardless of the thickness of the sample material and the laser power. An example of implementing a flat top profile is disclosed in DE 10 2011 116 405 A1 (corresponds to US 2013/0099112 A1). Using suitable specialized measures, it is also possible for the laser beam to have a Gaussian distribution.

The at least one modification to the extension of the beam impingement region is preferably made in such a way that the energy per area (fluence) acting on the sample material in the ablation area is kept substantially constant. In the case of a laser light beam, the fluence can be kept constant by coordinated adjustment of the focus size and the beam energy supplied by the laser resonator. Quick modulation of the beam energy can be achieved by, for example, an electro-optical component that preferably has a response time in the nanosecond range, e.g., an electro-optical modulator.

The sample material may be prepared with a light-absorbing matrix substance. For the ablation and/or desorption, the MALDI method may be possible in incident light (in reflection mode) or in transmitted light (in transmission mode), depending on the requirement. The MALDI method requires a certain sample preparation with a light-absorbent matrix substance, e.g., sinapic acid, 2.5-dihydroxybenzoic acid, α-cyano-4-hydroxycinnamic acid or 2.5-dihydroxyacetophenone, all of which absorb strongly in the ultraviolet spectral range. Laser light from a nitrogen laser with a wavelength of around 337 nanometers or from a frequency-tripled solid-state Nd:YAG laser at around 355 nanometers, for example, is suitable for the beam. The energy of the beam is preferably in the range of 0.01-50 microjoules; the lower limit can be applied particularly in the case of small laser foci on the sample material, as can be set with transmission MALDI, for example. Ablation and/or desorption can be supplemented by a post-ionization modality, such as the so-called MALDI-2 process (see Jens Soltwisch et al., Science, Mar. 5, 2015, Volume 348, Issue 6231, pages 211-215).

In various embodiments, the mass may be analyzed using a mass analyzer or coupled mobility mass analyzer. Ion-guiding intermediate stages, for example high-frequency voltage ion guides such as rod multipoles, stacked-ring ion guides or RF funnel arrangements, can be positioned upstream from the actual mass analyzer, or multiple analyzers in series, and also in different sections between such analyzers in series. Various analyzers and intermediate stages can also be operated at different vacuum levels.

A mass analyzer separates charged molecules or molecular ions according to their mass-to-charge ratio, usually designated as m/z. Time-of-flight analyzers can be used, for which both linear and reflector setups and/or setups with axial or orthogonal acceleration into the flight region can be chosen. Other types of mass-dispersive analyzers can also be used, e.g., quadrupole mass filters (single quads), triple quadrupole analyzers (“triple quads”), ion cyclotron resonance cells (ICR), Kingdon-type analyzers such as the Orbitrap® (Thermo Fisher Scientific), and others.

An ion mobility analyzer separates charged molecules or molecular ions according to their collision cross-section to charge ratio, sometimes designated by o/z or Q/z. The basis for this is the interaction between the ionic species and an electric field that couples with the charge of the ions, and the simultaneous effect of a buffer gas that interacts with the average cross-sectional area of the ion. Already known are, particularly, drift-tube mobility analyzers with static electric field gradients, which drive ions through an essentially stationary gas. Here, the drift velocity of an ionic species is given by the propulsive force of the electric field and the decelerating force of the collisions with the gas particles. Equally common are trapping ion mobility separators (TIMS) with a continuous laminar gas flow driving the ions forward, said gas flow being counteracted by a gradually changed electric field gradient with correspondingly variable deceleration force. Traveling-wave mobility analyzers are also worthy of mention. It is evident that analyzers and separators of the previously mentioned types can be coupled to enable ionic species to be separated multi-dimensionally, i.e., according to more than one physico-chemical property, such as m/z and σ/z or Ω/z.

A spectral dataset may comprise a frequency distribution of detected ionic species as a function of a mass parameter m or mass-related parameter such as m/z. A spectral dataset may be a spectrum but may also be a list of detected signals above ubiquitous noise (peak list). Depending on the analysis setup, a spectral dataset may contain information about the circumstances of data acquisition in the metadata. If, for example, a mobility analyzer is used before the actual mass analysis, the metadata of the spectral dataset can contain information about the analyzer setting at the time of data acquisition, possibly the measured value of mobility or of a related parameter such as collision cross section (CCS), elution voltage (especially with TIMS) or drift time. If, for example, a precursor ion selection by a mass filter and a fragmentation are carried out before a final mass analysis, the metadata of the spectral dataset, which would then essentially comprise signals from fragment ions, can contain information about the filter and fragmentation settings, e.g., bandpass width of the mass window and acceleration voltage for the collision-induced dissociation.

In various embodiments, the shape and/or outline of the ablation area and the shape and/or outline of the beam impingement region may differ substantially. The beam impingement region may have a circular shape and the ablation area can have a substantially different shape, e.g., an ellipse. In the example of an ellipse, a circle diameter can be determined that lies completely within the limits of the ellipse, e.g., if the circle diameter corresponds to the greatest possible distance between the opposite ellipse boundaries perpendicular to a line connecting the two focal points of the ellipse. However, given the requirement not to sample beyond the boundaries of the ablation area, crescent-shaped marginal areas of the elliptical shape remain unsampled when using such a central beam impingement region. For the purpose of further sampling of the ablation area, the circular beam impingement region may be modified, and, in particular, reduced in size so that it fits into the crescent-shaped boundaries of the marginal area without extending beyond the boundaries of the ablation area and without resampling already sampled partial sections, at least to a large extent. This can be achieved by suitably reducing the size of the beam impingement region and realigning the beam. If necessary, the change in the extension of the beam impingement region may also be accompanied by a change in the shape of the beam impingement region, e.g., from circular to oval or elliptical, etc.

In practice, it is irrelevant whether the largest possible beam impingement region is used first for sampling or one with smaller extension in comparison. By knowing the shape, outline and size of both the ablation area and the possible beam impingement regions, a work sequence for ablation and/or desorption can also begin with a smaller impingement region setting in the then theoretically determined, e.g., crescent-shaped, marginal areas. The central partial section of the ablation area would then be targeted with the enlarged and, in particular, largest possible impingement region setting. For post-processing and merging the molecular information into a spectral dataset, the sequence of the impingement region setting, e.g., from large to small or from small to large, is irrelevant during ablation and/or desorption.

In the example described above, the beam impingement region is assumed to be circular, and the surface of the ablation area is assumed to be elliptical. It goes without saying that the same considerations would apply if the surface of the ablation area were circular and the beam impingement region elliptical. The decisive factor is that the shapes and/or outlines differ substantially from one another, in particular that they are not substantially congruent, e.g., because at least one of the shapes and/or outlines does not originate from a standard geometric shape set (e.g., circle, square, rectangle, ellipse, etc.).

In various embodiments, the method may further comprise: Detecting a spatial distribution of a plurality of ablation areas on the sample support and determining an overall ablation area dimension which fits into each of the plurality of ablation areas, wherein the beam-assisted sampling and mass analysis are carried out discontinuously in that a first beam impingement region having first extension and then at least a second beam impingement region having second extension, which is different to the first extension, are applied to the plurality of ablation areas during the course of sampling, with the molecular information being obtained separately for each ablation area and later combined in a separate spectral dataset in each case. In this way, for example, a distribution of individual and isolated cells on the sample support can be sampled and mass analyzed efficiently and analyzer-friendly.

It is understood that principles of this disclosure are utilized when a single ablation area on a sample support is sampled by any of the methods described herein, even if more than one ablation area or a plurality of ablation areas are detected on the sample support. Principles of the disclosure are also realized when a certain proportion of ablation areas on a sample support are sampled by one of the methods described herein, whereas the remaining areas may be sampled by another method. In particular, according to the disclosure, the proportion cannot be greater than an upper limit selected from among the group including: 100% or less, 50% or less, 40% or less, 30% or less, 20% or less, 10% or less, 5% or less of all ablation areas identified and/or accessible on the sample support.

In various embodiments, the locating of the ablation area can be performed using an image recognition algorithm. In particular, the image recognition algorithm can be trained as artificial intelligence. The artificial intelligence may comprise an algorithm, in particular a self-adaptive algorithm, selected from among the group which includes: genetic algorithm, neural network, machine learning, e.g. a support vector machine (SVM), multi-layer machine learning (deep learning). An automated detection process speeds up data processing and may reduce the influence of subjective assessment.

In various embodiments, beam-assisted sampling may comprise relative movement between the beam impingement region and the sample support, which takes place without spatial adjustment of the sample support. The movement mechanism for beam movement, especially of a laser light beam, e.g., galvanometric mirrors, can be actuated quicker than a heavy translation stage that guides the sample support. Using the range of the beam alignment (movement radius), which can be up to a few millimeters on a sample support, sampling can be carried out responsively and fast.

In various embodiments, the method may also comprise: subjecting the molecular information and/or the spectral dataset to an analysis selected from among the group including: principal component analysis (PCA), cluster analysis, discriminant analysis, non-negative matrix factorization, rotation method. The aim of the analysis can be to localize and group the spectral data obtained in an abstract data space, and ultimately also to visualize it, e.g., on a computer screen, so that ablation areas with differing molecular information compositions can be distinguished from one another. The molecular information of an ablation area, e.g., of a single cell, can be compared with spectral data from a database for identification or classification purposes, as is common in so-called fingerprinting methods. Fingerprinting may target different biomolecules, e.g., peptides, proteins, lipids, (poly-) saccharides, metabolites, and others.

In various embodiments, sampling and mass analysis may be performed until (i) the sample material within the ablation area is ablated or desorbed over substantially the entire surface and/or completely or (ii) a signal quality parameter of the spectral dataset reaches or exceeds a threshold value. A signal quality parameter may, for example, comprise a minimum value for the total ion count (TIC) detected in the spectral dataset, an average ion current (e.g., arithmetic mean) or the median of the ion current, or in each case a predefined ion current interval, e.g., on a mass scale or mass-related scale. Ablation and/or desorption over the entire surface may involve sampling molecular information from each partial section of the ablation area without having to completely ablate and/or desorb the sample material. Complete ablation and/or desorption may mean that the sample material is so completely ablated and/or desorbed over the ablation area that the sample support shines through. Complete ablation and/or desorption may be detected by the real-time detection of ionic species originating from the surface of the sample support, e.g., metal atoms, and then lead to the termination of ablation and/or desorption from the ablation area, or a partial section thereof.

An alternative embodiment monitors the molecular content of each individual ablation operation from a series of ablation operations from the same (partial) ablation area and terminates sampling from that (partial) ablation area if no molecular content significantly distinguishable from noise is discernible in the spectral data. For certain applications, a condition may be defined according to which the detection of a certain signal in the acquired spectral data in real time determines whether sampling of an ablation area is continued or not. A biomarker in the form of a specific mass signal or a specific mass signal signature can, for example, indicate pathologically modified tissue. Cancerous tissue (cancer) is one example. At the point in time when continued sampling allows the biomarker to be reliably detected above the noise, sampling can be stopped and continued at another sampling area, if there is one.

According to a further aspect, this disclosure relates to a device for spectrometric analysis of sample material located in an ablation area on a sample support, comprising: —a non-mass analytical imaging device arranged and designed to detect and localize ablation areas on the sample support; —a beam device arranged and designed to locally impact sample material on the sample support and to adjust a beam impingement region; —a first positioning device arranged and designed to align and move the beam impingement region on the sample support; —a second positioning device arranged and designed to spatially adjust the sample support; —a mass analyzer arranged and designed to receive and process ablated and/or desorbed and ionized sample material from the sample support; —a digitizing unit arranged and designed to convert analog molecular information into digitized molecular information and to output it as a spectral dataset; and —a control system which communicates with the imaging device, the beam device, the first positioning device, the second positioning device, the mass analyzer and the digitizing unit, and is programmed and designed to carry out a method as described herein above.

In various embodiments, the non-mass analytical imaging device may be non-sample consuming and, in particular, may be selected from among the group including: microscopy, interferometry (white light), magnetic resonance imaging (with micrometer resolution). Microscopy may take the form of bright-field microscopy, dark-field microscopy, infrared microscopy, phase-contrast microscopy, near-field microscopy, polarization microscopy and/or fluorescence microscopy. The fluorescence can be autofluorescence or excited fluorescence. The sample material may be treated with a suitable fluorescent dye prior to image acquisition using the non-mass-analytical imaging modality. The dye treatment may be applied locally to the ablation areas so that other areas remain untreated. An example of a fluorescent dye is Hoechst 33342 (bisbenzimide), which is excited by ultraviolet light at a wavelength of about 340 nanometers (unbound) or 355 nanometers (bound) and emits light in the spectrum from blue to cyan (˜510 nanometers). Bound to deoxyribonucleic acid, the immission maximum is 355 nanometers; the emission maximum is around 465 nanometers. Wheat germ agglutinin (WGA) may also be used to examine individual cells and, in particular, the cytoplasm and/or the cell membrane. It can be combined effectively with another dye for the core, e.g., the aforementioned Hoechst 33342 or 4′,6-diamidin-2-phenylindole (DAPI). Individual cells may be easily segmented following appropriate treatment.

The beam device may comprise adjustable beam-directing elements that are controlled to adjust the beam direction. Preferably, the beam-directing elements have galvanometer micromirrors for a light beam or pairs of opposing DC electrodes for a primary ion beam, e.g., for an ionization process according to secondary ion mass spectrometry (SIMS). For a light beam such as a laser beam, an arrangement of reflective elements such as mirrors, refracting elements such as prisms, and/or electrooptical or acoustooptical deflection elements such as electrooptical crystals, can be used, in particular, and this arrangement is able to change or shift a position where the ablating and/or desorbing beam impinges on the sample support, or on the sample material on the support, in two spatial directions along a plane parallel to the sample support surface, e.g., by tilting or otherwise changing the spatial orientation. For a primary ion beam, a system of opposing deflection electrodes supplied with a potential can be used, which surround the primary ion beam path and can generate variable potential gradients in the space between the two opposing electrodes of a pair. An example is two opposing electrode pairs, at 90° to each other. If the electric potential applied to the two opposing electrodes of a pair is the same, an ion beam passing through this pair will not experience any lateral deflection. If the electric potentials are not the same, the primary ions are deflected as a function of the potential difference during flight.

The first positioning device may adjust the beam impingement region, e.g., an enlargement or reduction in size, using optics comprising a variable focal length objective. It is also possible to move the sample support out of the focal plane of the beam, which regularly marks the point of least spatial beam expansion along its path, using the appropriate movement mechanism. This approach would increase the size of the impingement region. Of course, it is also conceivable to change both the focal length of an objective forming the beam and the position of the sample support along a direction perpendicular to the sample support surface in order to produce a desired modification to the impingement region.

It is also possible to work instrumentally with a fixed number of preset and selectable beam impingement region extensions. This discretizes, simplifies and accelerates the modification to the beam impingement region. It is possible, for example, to provide a plurality of parallel beam paths between the beam generator (e.g., laser resonator) and the sample support, each of which has specific beam shaping and/or beam conditioning modules and may be selected, for example, using a very fast-responding (e.g., electro-optical) switch. In general, the number of parallel beam paths for different beam versions may be selected from among the group including: ten, nine, eight, seven, six, five, four, three and two beam paths. The preset beam impingement regions may, in particular, include dimensions, e.g., with regard to circle diameter or edge length, which are selected from among a group including: 50, 30, 20, 15, 10, 5, 4, 3, 2 and 1 micrometer, or any other scalar value between 1 and 50 micrometers. It is understood that the energy per area of the beam on the sample material or sample support is preferably kept substantially constant when the beam impingement region is modified. The selection of a beam path with certain preset extension of the beam impingement region is then accompanied by a corresponding adjustment of the beam energy supplied by the beam generator, e.g., a laser resonator.

It is also possible to provide a plurality of parallel beam paths between the beam generator and the sample support, each beam path from the plurality of beam paths having beam shaping and conditioning modules, of which at least one module of at least one beam path of the plurality of beam paths is equipped with a mechanism for variable adjustment of the extension of the beam impingement region. In the case of laser light, the variable adjustment module may comprise, for example, an objective with a variably adjustable focal length, in particular, using coordinated axial displacements of the imaging lenses. In this way, the non-utilization of this beam path from the plurality of beam paths can be used to adapt the beam impingement region that results when the beam path is used with the extension modification mechanism to suit the ablation area requirements. Such alternating use of parallel beam paths with at least partially adjustable beam impingement regions can accelerate the sampling and mass analysis of sample material from ablation areas.

The second positioning device may be arranged and configured to modify the position of the sample support in an xy-plane which is essentially perpendicular to a surface normal of the sample support. Preferred second positioning devices are xy translation stages or comparable actuators designed to adjust or move the sample support deposited on or in them, together with the sample material, in two spatial directions parallel to a plane of the sample support surface. The larger the movement radius of the first positioning device for modifying the alignment of the ablating and/or desorbing beam, the smaller the requirements for the smallest step width of a translation stage may be. The movement radius of the first positioning device may correspond to an upper limit selected from among a group including: 10 millimeters or less, 5 millimeters or less, 3 millimeters or less, 2 millimeters or less, 1 millimeter or less. Conventional step sizes of a traditional translation stage with stepper motor may be in the range of two-digit to three-digit micrometer intervals. Piezo-electrically operated translation stages from the manufacturer SmarAct (Oldenburg, Germany), in particular, the CLL, CLS and SLC series, may be mentioned.

The movement mechanism for the sample support, particularly implemented in or on the translation stage, can also include the possibility of adjustment along a third spatial direction (z-axis), for example to adapt an optimum focal point of an ablation and/or desorbing beam on sample material that varies in thickness or height, as described in the patent publication DE 10 2007 006 933 A1 (corresponds to US 2008/0191131 A1) of the applicant. When irradiating at an angle, this z-adjustment also results in an adjustment in the spatial directions xy perpendicular to it (i.e., parallel to the sample support surface), which can be compensated for by adjusting the beam alignment.

The digitization unit may have an analogue-to-digital converter with a dynamic range of 10 bits or 14 bits, for example. The control system may be designed as a processor, microprocessor or comparable computer and may contain or access program code with which the methods described herein above can be executed.

### BRIEF DESCRIPTION OF THE ILLUSTRATIONS

The invention can be better understood by referring to the following illustrations. The elements in the illustrations are not necessarily to scale, but are primarily intended to illustrate the principles of the invention (mostly schematically). In the illustrations, the same reference numbers designate corresponding elements in the different views.

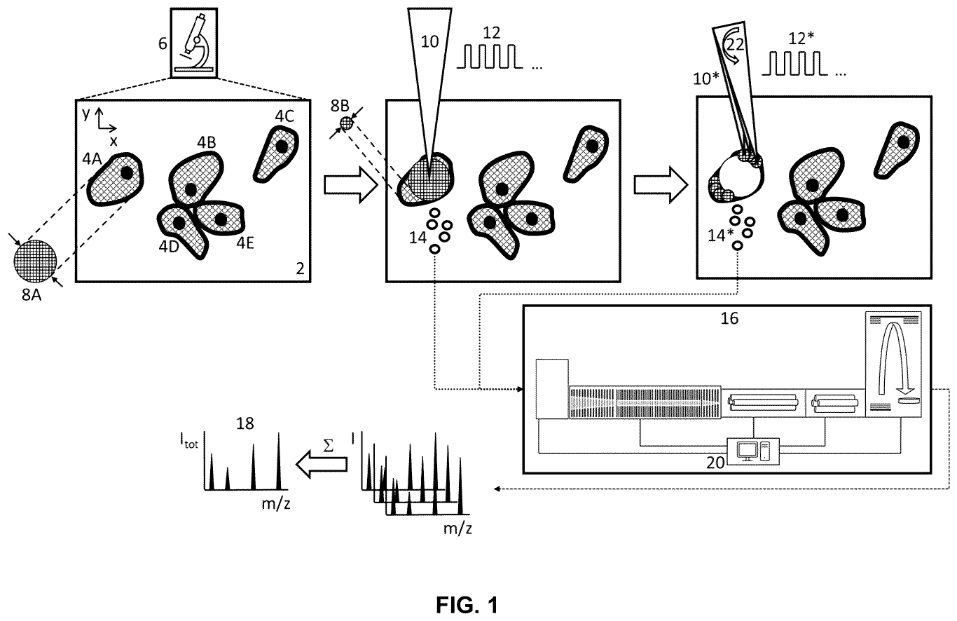

FIG. 1 illustrates, by way of example, a procedure for spectrometric analysis of sample material according to principles of this disclosure in several steps.

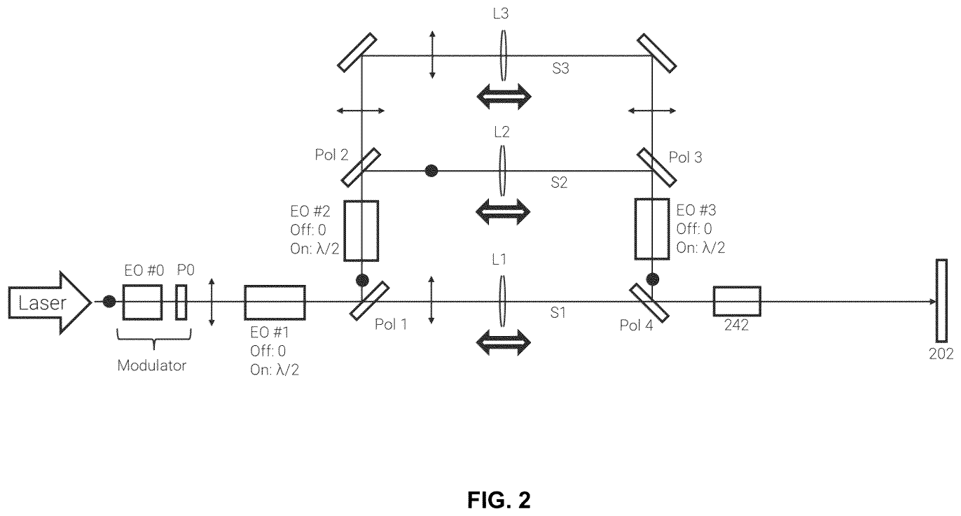

FIG. 2 shows a setup for a plurality of parallel beam paths in order to achieve a change in the extension of the beam impingement region in a time-saving manner.

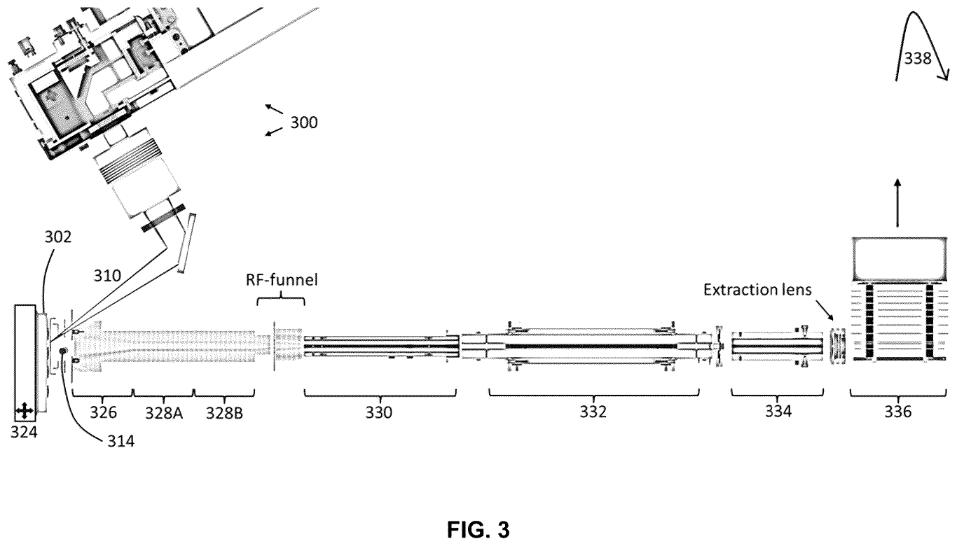

FIG. 3 shows an example of the design and operation of a combined mobility-mass analyzer for which ions are generated using matrix-assisted laser desorption and ionization (MALDI).

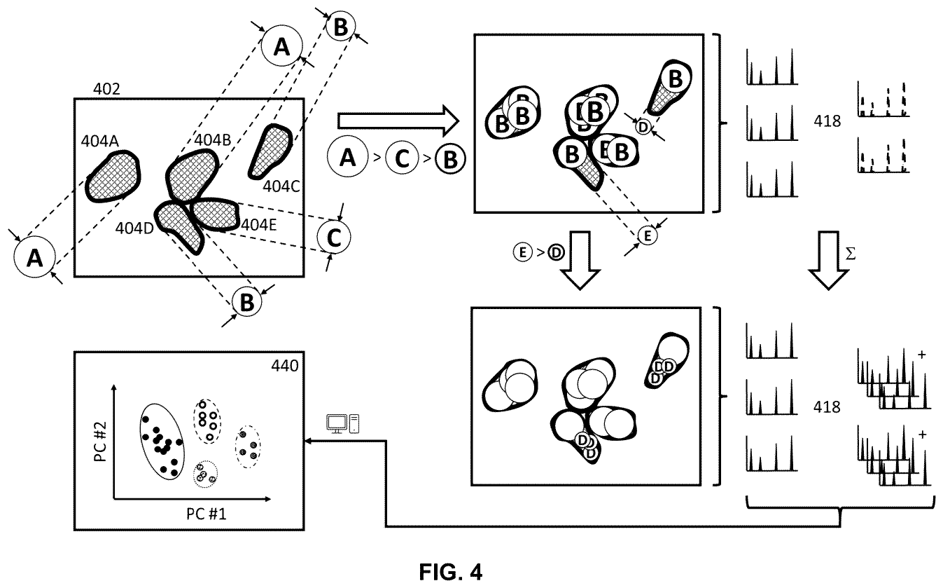

FIG. 4 illustrates, by way of example, a further procedure for spectrometric analysis of sample material according to principles of this disclosure.

### DETAILED DESCRIPTION

While the invention has been illustrated and explained with reference to a number of embodiments thereof, those skilled in the art will recognize that various modifications in form and detail can be made without departing from the scope of the technical teaching, as defined in the attached claims.

FIG. 1 shows, by way of example, a procedure for spectrometric analysis of sample material according to principles of this disclosure in several steps.

Sample material is arranged on a sample support (2). The sample material can be divided into five distinct areas (4A, 4B, 4C, 4D, 4E), as shown. These may, for example, be single cells that were grown directly on the sample support (2) or applied to the sample support (2). The single cells may contain nuclei. Alternatively, they may be sections in a larger piece of sample material, e.g., cell areas in a tissue.

Using a non-mass-analytical imaging modality, the ablation areas (4A, 4B, 4C, 4D, 4E) are localized and identified on the sample support (2). A possible design for such a multimodal analyzer is explained in the parallel application PCT/DE2022/100627 of the applicant. Localization may consist in determining the position coordinates of the various ablation areas (4A, 4B, 4C, 4D, 4E) on the sample support (2). The non-mass analytical imaging modality may include a microscopic technique, e.g., bright-field microscopy, dark-field microscopy, infrared microscopy, near-field microscopy, polarized-light microscopy, phase contrast microscopy and/or fluorescence microscopy (schematically at (6)). The boundaries of the ablation areas (4A, 4B, 4C, 4D, 4E) are defined in the recorded image of the imaging modality. This can be done automatically by image recognition algorithms, possibly using artificial intelligence, or manually by a user on a computer-generated representation of the image.

When the boundaries of the ablation areas (4A, 4B, 4C, 4D, 4E) have been determined, the extension for an impingement region (8A) that a beam (10) can encompass for the ablation and/or desorption of sample material from an ablation area (4A) without exceeding the ablation area boundaries can be determined. Preferably, the extension of the beam impingement region (8A) is selected so that the beam (10) impinges the largest possible contiguous partial section of the ablation area (4A). If a plurality of beam paths is used between the beam generator (not shown) and the sample support (2), a fixed setting of the beam impingement region may be selected that comes closest to the determined dimension and fits into the ablation area (4A). It is therefore not essential to select the mathematically largest possible beam impingement region as in the illustrated example, in which an ideal beam impingement region can be infinitely adjusted. The sample material in the partial section of the ablation area (4A) may be exposed to a plurality of pulses of coherent light (12) originating from a laser resonator. To improve the ablation and/or desorption and ionization of the sample material, the sample material in the ablation areas (4A, 4B, 4C, 4D, 4E) may be treated with a light-absorbing matrix substance for matrix-assisted laser desorption and ionization prior to exposure. Each pulse (10) ablates and/or desorbs sample material, and this is usually accompanied by ionization processes caused by charge carrier transfer. If the charge carrier supply for ionization during or after ablation and/or desorption does not meet the requirements for a usable signal quality, post-ionization modalities may be used, for example the so-called MALDI-2 process.

The ablated and/or desorbed and ionized sample material (14) is fed to a gas phase ion analyzer (16) via an interface. The gas-phase ion analyzer (16) may comprise one or more stages in which gas-phase ions are sorted according to their mass-to-charge ratio m/z or their cross-section-to-charge ratio σ/z or Ω/z. Time-of-flight analyzers are particularly preferred for the final generation of a spectral dataset or mass spectrum (18), as they can analyze ion ensembles in rapid succession and also cover a wide mass range. Gas-phase ions generated by repeated pulses (12) of the beam (10) on the partial section of the ablation area (4A), are passed through the ion analyzer (16) and finally collected or added up in a single spectral dataset or spectrum (18), and then output and stored via a data processing system (20).

To illustrate principles of this disclosure, the beam impingement region (8A) determined for the selected ablation area (4A) is circular in this example, whereas the shape of the available ablation areas (4A, 4B, 4C, 4D, 4E) on the sample support is not circular, but rather elongated and contains partly asymmetrically tapering partial sections. These partial sections cannot be impinged with the first selected setting of the beam impingement region (8A) without exceeding the ablation area boundaries. In the example shown, approximately crescent-shaped marginal areas above and below the first sampled partial section remain untouched by the beam (8A). It may be deemed necessary to sample the surface of an ablation area (4A) as completely as possible, either to maximize the signal height in the spectral dataset (18) or to sample and analyze the complete molecular information of the entire ablation area (4A), for example if this molecular information has a composition that is unevenly distributed within the boundaries of the ablation area (4A). An example of this is the molecular information from a cell nucleus and the molecular information within the cell from the area surrounding the nucleus, which can differ significantly from each other.

A modification to the initial beam impingement region (8A) is derived from the crescent-shaped marginal ablation areas. In the example shown, this modification may consist of a reduction in the diameter of the circular area (8B). The modified extension is preferably selected so that the beam impingement region with modified extension (8B) can impinge as large a partial section of the crescent-shaped areas as possible without touching too much of the already sampled partial sections. Here too, a possible alternative is to select a fixed beam impingement region setting, from a plurality of beam impingement region settings, which fits into the partial sections, even if it does not correspond to the largest possible beam impingement region for these partial sections. By quickly controlling the ablating and/or desorbing beam (10*), the impingement region (8B) can be adjusted across the marginal areas within the boundaries of the ablation area (schematically at (22)), with different pulses (12*) of the beam (10*) being triggered. The individual pulses (12*) may impinge slightly overlapping partial sections in the marginal area of the ablation area (4A). The sample support (2) remains in the originally adopted position during this continued sampling. Adjustment of a translation stage (not shown) guiding the sample support (2) is only necessary if the movement radius of the modification of the beam alignment (10*) is exhausted. An example of such an approach consisting of combined and alternating stage movement and beam scanning is disclosed in DE 10 2021 114 934 A1 (corresponds to US 2022/0397551 A1) of the applicant.

One requirement for sampling can be to ablate and/or desorb the sample material largely over the entire surface and/or completely within the boundaries of the ablation area (4A) until the bare sample support (2) shows through. Of course, it is efficient if the areas of individual pulses (12*) do not overlap too much. The ionized sample material (14*) from the marginal areas, which has been transferred to the gas phase, is fed to the ion analyzer (16), as before, and sorted and detected according to its physico-chemical properties. The molecular information is then added to the spectral dataset (18) so that a single, standardized spectral dataset (18), e.g., in the form of a spectrum or spectra, is available for an ablation area (4A).

The procedure described enables better utilization of the dynamic range of an ion detector with a connected digitization unit, since fewer spectra are added up per scanning range (4A, 4B, 4C, 4D, 4E), so noise background in the individual spectra in the total spectrum (18) is less significant. Furthermore, the load on the movement mechanics of the beam (10, 10*) is relieved because fewer alignment modifications to the beam (10, 10*) have to be made during the sampling of an ablation area (4A, 4B, 4C, 4D, 4E). This improvement is somewhat weakened by the fact that the beam impingement region (8A=>8B) must also be modified by drives. However, it is to be expected that only in exceptional cases will this lead to a zero-sum game for the loads on the respective mechanisms.

Instead of recording a plurality of spectra from partial sections from the ablation area, which are then mathematically combined into an overall spectrum of the ablation area during post-processing, an ion buffer can also be provided for the uninterrupted sampling of a single ablation area, as shown in FIG. 1, e.g. a linear or two-dimensional RF multipole ion trap, in which the ablated and/or desorbed and ionized sample material of the ablation area is collected and, when the ablation and/or desorption of the ablation area is complete, is transferred to the connected ion analyzer.

FIG. 2 shows a setup with three selectable parallel beam paths (S1, S2, S3), which can be used to generate different extensions of beam impingement regions of a laser light beam in a very flexible and time-saving manner:

The pre-attenuated, linearly polarized pulsed laser beam passes via a modulator (EO #0) and a polarizer (P0), which together form a modulator for attenuation, and a first electro-optical crystal (EO #1) into a first beam branch.

The linear polarization state of the laser light can lie in the display plane (represented by arrow) or oscillate perpendicular to the display plane (represented by dot).

The electro-optical crystal (EO #0) of the modulator changes the polarization state of the laser beam, depending on the voltage applied. The downstream polarizer (P0) is set vertically with respect to the polarization of the incident light. This means that the laser light is almost completely blocked when no voltage is applied to the electro-optical crystal (EO #0).

Given the pulsed laser beam used here with pulse widths in the nanosecond range, a voltage pulse with a temporal length greater than the pulse width of the laser beam can be applied at the time it passes through the modulator. This applied voltage leads to partial transmission through the modulator, allowing the strength of the laser beam to be modified for each laser pulse. The crystal can be operated with response times in the nanosecond range, so even at high repetition rates of the laser in the range of several tens of kilohertz, the strength of each laser pulse can be adjusted individually.

The polarized laser light, whose intensity has been adjusted, then enters a first electro-optical crystal (EO #1). A voltage applied at the time the laser pulse passes through the first crystal (EO #1) determines the further path of the laser pulse. If no voltage is applied, the laser beam passes through a first polarizer (Pol 1), through a first lens (L1), and through a fourth polarizer (Pol 4) into an objective (242). The objective (242) focuses the laser beam onto the sample material or a sample support (202) on which the sample material has been placed. This comprises the first beam path (S1). The long focal length of the first lens (L1) results in a slight defocusing of the laser beam on the sample material or sample support (202).

If a λ/2 voltage is applied to the first electro-optical crystal (EO #1), the polarization of the laser pulse is rotated by 90° (arrow to dot representation) and thus deflected in the direction of a second electro-optical crystal (EO #2).

If no voltage is applied to the second electro-optical crystal (EO #2), the laser pulse passes via a second polarizer (Pol 2) to a lens (L2), is deflected by a third polarizer (Pol 3), passes into a third electro-optical crystal (EO #3) and is deflected via the fourth polarizer (Pol 4) into the objective (242), provided that no voltage is applied to the third electro-optical crystal (EO #3). This comprises the second beam path (S2).

If a λ/2 voltage is applied to the first, second and third electro-optical crystals (EO #1, EO #2, EO #3), the laser pulse travels to the objective via two beam deflecting elements, e.g., mirrors, and a third lens (L3). This comprises the third beam path (S3).

By selecting different focal lengths and/or positions of the first, second and/or third lenses (L1, L2, L3), defocusing on the sample material or sample support (202), and thus the size of the beam impingement region, can be adjusted by selecting the voltage state on the first, second and/or third electro-optical crystals (EO #1, EO #2, EO #3) at the time of beam passage.

This allows three different beam impingement region extensions or beam diameters to be realized on the sample material or sample support (202) from shot to shot:

| Lens # | Voltage EO #1 | Voltage EO #2 | Voltage EO #3 |
|---|---|---|---|
| 1 | Off | Off | Off |
| 2 | On | Off | Off |
| 3 | On | On | On |

The number of parallel beam paths is not limited and can be modified or extended accordingly. The illustration shows an example with three beam paths (S1, S2, S3). Of course, two, four or more parallel beam paths are also possible. The set-up shown must then be adjusted accordingly, e.g., by adding appropriate electro-optical crystals.

With fixed positions for the first, second and third lenses (L1, L2, L3), it is possible to switch quickly between various predefined beam impingement regions or focal points. If one or more beam paths are not used for a prolonged period of time, the beam impingement region or focusing can be reconfigured during this time by mechanically moving the corresponding lenses arranged in the non-selected beam path (indicated by thick double-headed horizontal arrows). Therefore, more options are available for adjusting the beam impingement region or focus than the number of beam paths alone would suggest.

For further classification of the disclosure, FIG. 3 provides a schematic representation of a possible setup of an ion analyzer, as outlined at (16) in FIG. 1, wherein the analyzer connected via an interface is a so-called TIMS mobility mass spectrometer (adapted from Jeffrey M. Spraggins et al, Anal Chem. 2019 Nov. 19; 91(22) 14552-14560). The setup and operation will be explained very briefly:

A laser system (300), as a beam device and first positioning device with various light-optical beam guidance elements, is designed to bombard sample material on a sample support (302) in pulses. The beam alignment may be modified step by step using fast-reaction components such as galvanometric micromirrors in order to target different impingement points on the sample material and/or on the sample support (302). The laser system (300) also includes components that allow a focus size of the laser beam to be modified on the sample support (302), e.g., an objective with variable focal length on short time scales (not shown) or a setup as discussed with reference to FIG. 2.

The sample support (302) is coupled to a second positioning device (324), e.g., placed on a translation stage, which can be adjusted in one, two or even three spatial directions (xyz) in order to approach different areas on the sample support (302) to be targeted by a laser beam (310).

A control system (not shown), suitable for implementing principles of this disclosure and programmed accordingly, communicates with the adjustable beam-guiding elements in the laser system (300) and the second positioning device (324), and coordinates the operation of both. The impingement areas on the sample support (302) are adjusted so that the sample material (314) ablated and/or desorbed and ionized by the beam (310) is taken up by an interface in the form of an RF ion funnel (326) and spatially collimated and compacted into a thin ion filament for onward transmission through the arrangement. In the arrangement shown, a surface normal to the sample support (302), which originates from the set impingement area, runs through the openings in the electrodes of the RF ion funnel (326). The electrode module of the first funnel element, or more precisely its opening, sets the framework of geometric acceptance for the reception and forwarding of locally ablated and/or desorbed and ionized sample material (314).

After it has been generated, the charged sample material (314) passes through the RF ion funnel (326) and enters a mobility analyzer, which has an accumulating region (328A) and a subsequent analyzing region (328B). The principle of such a dual design is described, for example, in the patent publication US 2016/0231275 A1 of the applicant. An inert gas flows through both regions of the mobility spectrometer (328A, 328B) (from left to right in the illustration). The charged sample material (314) is driven within it by the gas flow against an opposing electric field. In the analyzing area (328B), the charged sample material (314) is separated and stored at different positions along the horizontal axis, depending on the respective mobility.

Incremental reduction of the electric field strength in the analyzing region (328B) of the mobility analyzer enables sequential delivery of charged sample material (314), separated by mobility. After mobility analysis in the analyzing region (328B), the charged sample material (314) that has collected meanwhile in the accumulating region (328A) is transferred to the analyzing region (328B). The charged sample material (314) exiting the analyzing region (328B) first passes through a transfer multipole (330) and then enters a quadrupole mass filter (332). Here, the charged sample material (314) can be selected for further analysis, while other charged sample material can be filtered out. Subsequently, the charged sample material (314) is transferred to a collision cell (334), where the selected charged sample material can be fragmented by accelerated injection into a neutral gas. The collision cell (334) can, of course, also be operated in such a way that substantially no fragmentation of the charged sample material (314) takes place, for example, for the purpose of conducting a survey measurement of precursor compounds.

In the collision cell (334), the charged sample material (314) and/or any charged fragments generated from it are temporarily stored before being introduced in a synchronized manner into the pulser (336) of a time-of-flight analyzer with orthogonal injection. There it is accelerated, perpendicular to the direction of entry, onto the flight path of a reflector time-of-flight analyzer. At the end of the flight path, after the direction of flight is reversed in a reflector (indicated by arrow (338)), a detector (not shown) receives the various time-resolved and thus mass-resolved packets and outputs them as a time-of-flight transient, which can subsequently be converted into masses (m) or mass/charge ratios (m/z).

FIG. 4 shows, by way of example, a further procedure for spectrometric analysis of sample material according to principles of this disclosure in several steps.

Instead of considering the sampling of a single ablation area (404A) as a continuous, uninterrupted process, in this embodiment a plurality of ablation areas (404A, 404B, 404C, 404D, 404E) are addressed for a particular execution of discontinuous, multiplexed processing. First, the ablation areas (404A, 404B, 404C, 404D, 404E) are localized and delineated on a sample support (402), e.g., using a non-mass-analytical imaging modality, as previously explained. Using powerful image recognition algorithms, an optimum beam impingement region (A, B, C) can be determined for each ablation area (404A, 404B, 404C, 404D, 404E) so that it fits within the boundaries of the respective ablation area (404A, 404B, 404C, 404D, 404E) and covers the largest possible partial section. For a multiplexed analysis, the impingement region (A, B, C) is then selected that is compatible with the shapes and sizes of all ablation areas (404A, 404B, 404C, 404D, 404E) without exceeding the respective ablation area boundaries. In the illustrated example, this is initially the impingement region (B). As explained above, instead of a mathematically ideally adapted beam impingement region, a fixed beam impingement region setting can also be selected which does not correspond to the mathematically largest possible setting and still fits into all ablation areas (404A, 404B, 404C, 404D, 404E), e.g., using a plurality of parallel beam paths between the beam generator and the sample support (402).

With the selected impingement region setting (B), the ablation areas (404A, 404B, 404C, 404D, 404E) are sampled for as long as possible or necessary. In the example shown, it turns out that the selected setting (B) is dimensioned against the background of the shapes and sizes of the ablation areas (404A, 404B, 404C, 404D, 404E) so that three of the ablation areas (404A, 404B, 404E) can already be sampled over the entire surface. Two of the sampling areas (404C, 404D) can only be sampled in partial sections, so marginal areas remain unsampled. While the molecular information of the three ablation areas (404A, 404B, 404E) sampled over the entire surface is summed up in separate individual spectral datasets (418), the next step is to examine the partial sections at the margins and to determine a setting for the beam impingement region (E, D) that allows these partial sections at the margins to be sampled without exceeding the boundaries. This can be a mathematically ideally selected and instrumentally set up impingement region or a specific suitable beam impingement region from a plurality of predefined and selectable beam impingement regions. In the illustrated example, this is the impingement region (D).

The spectral datasets (418), which are still incomplete with regard to the molecular information, are completed by applying a suitable impingement region setting (D) to the marginal partial sections of the two ablation areas (404C, 404D) by addition and/or summation. The completed individual separate spectral datasets (418) obtained from the respective ablation areas (404A, 404B, 404C, 404D, 404E) can then be subjected to multivariate statistical analysis (schematically at (440)), such as principal component analysis, to locate and differentiate the molecular information in an abstract data space. Such an evaluation should make it possible to distinguish spectral data (418) from ablation areas with sample material that have different molecular information compositions. Alternatively, it is possible to output the obtained spectral data as images in which the scanning areas, e.g., individual cells, do not consist of many pixels, but each scanning area, in particular irregularly shaped scanning areas, is a color-individualized pixel that is co-registered with an image of an optical or microscopic modality, for example, and is displayed superimposed.

In the examples described above, a larger impingement region was first used for sampling, and then a comparatively smaller impingement region was used. This sequence is not mandatory. It is also possible to start with a small setting, with which partial sections of the ablation areas are impacted, which are arranged around an area that can be sampled with the largest possible, or largest possible selectable, beam impingement region. This does not adversely affect the calculation of the respective partial spectral data for the final creation of a single overall spectral dataset per ablation area.

In the examples explained, ablation areas or regions of interest are sampled with two different beam impingement regions. Of course, it is possible to take this express disclosure further in such a way that more than two different beam impingement regions are also selected, e.g., three, four, five, etc., in order to sample one or more ablation areas, provided that such an approach is appropriate to the application and appears reasonable to the person skilled in the art.

The invention has been described above with reference to several specific example embodiments. It is to be understood, however, that various aspects or details of the embodiments described can be modified without deviating from the scope of the invention. Furthermore, the features and measures disclosed in connection with different embodiments can be combined as desired if this appears practicable to a person skilled in the art. Moreover, the above description serves only to illustrate the invention and not as a limitation of the scope of protection, which is exclusively defined by the appended Claims, taking into account any equivalents which may exist.

1. A method for the spectrometric analysis of sample material located in an ablation area on a sample support, comprising:
localizing the ablation area on the sample support and determining an ablation area dimension between opposite boundaries of the ablation area;
beam-assisted sampling from the ablation area and mass analysis of the ablated and/or desorbed and ionized sample material, wherein a beam impingement region, which is selected no larger than the ablation area dimension, is moved within the boundaries of the ablation area while performing ablation and/or desorption operations and an extension of the beam impingement region is changed at least once; and
combining the molecular information obtained from the ablation and/or desorption operations from the ablation area into a single spectral dataset.

2. The method according to claim 1, wherein a single cell on the sample support is selected as the ablation area.

3. The method according to claim 1, wherein the ablation area dimension corresponds to the largest possible extension of the beam impingement region on the sample material in the ablation area.

4. The method according to claim 1, wherein a shape and/or outline of the ablation area and a shape and/or outline of the beam impingement region differ substantially.

5. The method according to claim 1, further comprising: detecting a spatial distribution of a plurality of ablation areas on the sample support and determining an overall ablation area dimension which fits into each of the plurality of ablation areas, wherein the beam-assisted sampling and mass analysis are carried out discontinuously in that a first beam impingement region having first extension and then at least a second beam impingement region having second extension, which is different to the first extension, are applied to the plurality of ablation areas during the course of sampling, with the molecular information being obtained separately for each ablation area and later combined in a separate spectral dataset in each case.

6. The method according to claim 1, wherein the ablation area is localized using an image recognition algorithm.

7. The method according to claim 6, wherein the image recognition algorithm is trained as artificial intelligence.

8. The method according to claim 1, wherein the beam-assisted sampling comprises a relative movement between the beam impingement region and the sample support, which takes place without spatial displacement of the sample support.

9. The method according to claim 1, further comprising subjecting the molecular information and/or the spectral dataset to an analysis selected from among the group including: principal component analysis (PCA), cluster analysis, discriminant analysis, non-negative matrix factorization, rotation method.

10. The method according to claim 1, wherein the sampling and mass analyzing are performed until (i) the sample material within the ablation area is ablated and/or desorbed substantially over the entire surface and/or completely or (ii) a signal quality parameter of the spectral dataset reaches or exceeds a threshold value.

11. A device for the spectrometric analysis of sample material located in an ablation area on a sample support, comprising:
a non-mass-analytical imaging device which is set up and designed to detect and localize ablation areas on the sample support;
a beam device which is set up and designed to locally impact sample material on the sample support and to adapt a beam impingement region;
a first positioning device which is set up and designed to align and move the beam impingement region on the sample support;
a second positioning device which is set up and designed to spatially adjust the sample support;
a mass analyzer which is set up and designed to receive and process ablated and/or desorbed and ionized sample material from the sample support;
a digitizing unit which is set up and designed to convert analog molecular information into digitized molecular information and to output it as a spectral dataset; and
a control system which communicates with the imaging device, the beam device, the first positioning device, the second positioning device, the mass analyzer and the digitizing unit, and is programmed and designed to carry out a method according to claim 1.

12. The device according to claim 11, wherein the non-mass analytical imaging device is non-sample consuming, and, in particular, is selected from among the group including: microscopy, interferometry, magnetic resonance imaging.
